(19) United States
(12) Patent Application Publication
(10) Pub. No.: US 2013/0096447 A1
(43) Pub. Date: Apr. 18, 2013

(54) System and methods for serial analysis of electrocardiograms

(71) Applicants: Akshay Dhawan, Randolph, NJ (US); Brian Wenzel, San Jose, CA (US); Samuel George, Portland, OR (US); Bosko Bojovic, Beograd (SE); Ihor Gussak, Morristown, NJ (US); Dorin Panescu, San Jose, CA (US)

(72) Inventors: Akshay Dhawan, Randolph, NJ (US); Brian Wenzel, San Jose, CA (US); Samuel George, Portland, OR (US); Bosko Bojovic, Beograd (SE); Ihor Gussak, Morristown, NJ (US); Dorin Panescu, San Jose, CA (US)

(21) Appl. No.: 13/573,593

(22) Filed: Sep. 27, 2012

Related U.S. Application Data
(60) Provisional application No. 61/626,533, filed on Sep. 27, 2011.

(51) Int. Cl. A61B 5/04

(52) U.S. Cl. CPC A61B 5/04017
USPC 600/512

(57) ABSTRACT

Systems and methods for serial analysis of electrocardiograms are presented, wherein serial electrocardiographic (ECG) assessment is incorporated with three-dimensional vectorial analysis of the cardiac electrical signal, using changes in novel 3D-based vectorial markers over time to improve diagnostic sensitivity for acute coronary syndromes (ACS), and improve differentiation of ACS from the broad range of heart diseases that resemble ACS on ECG.

### CROSS-REFERENCE TO RELATED APPLICATIONS

The present invention claims priority under 35 U.S.C. 119 to U.S. Provisional Patent Application No. 61/626,533, filed Sep. 27, 2011, incorporated by reference in entirety.

### FIELD OF THE INVENTION

The invention relates generally to the analysis of electronic cardiac signals for use in clinical diagnostics, and specifically to systems and methods configured to assist in the analysis of details of ECG signals and vector cardiograms to determine how patients should be categorized into specific cardiac risk categories, such as an acute coronary syndrome category.

### BACKGROUND

Approximately 6.5 million patients present to U.S. emergency departments (“ED”) each year with chest pain. With the benefit of retrospective study, it is apparent that approximately 5.4 million of those patients do not have acute coronary syndrome (“ACS”), but rather some other clinical condition, such as heartburn, gall stones, or the like. Of the approximately 5.4 million, about 26% have ACS ruled out by a first diagnostic triage in the ED, typically comprising at least a 12-lead electrocardiogram (or “ECG” or “ECG”) study and blood troponin levels (a biomarker for cardiac injury). The remaining 74% of these 5.4 million patients are kept around in the hospitals for cardiac additional testing, until it is subsequently discovered, through additional time and testing, that most of these patients do not suffer from ACS.

Most commonly, the initial ECG in possible ACS is nondiagnostic, and additional workup is needed. Such additional workup (which may include, for example, ultrasonography of the heart, cardiac nuclear imaging, or invasive cardiac catheterization) is expensive and time-consuming. Moreover, it is not uncommon that diagnostic uncertainty results either in unnecessary hospitalization of the patient, or the incorrect discharge of a patient who in fact has true ACS. Both are highly undesirable outcomes that lead to higher healthcare costs or poor clinical outcomes.

Repeated assessment of ECGs over time (sometimes referred to herein as “serial” or “dynamic” ECG analysis) has the potential to improve accuracy and timeliness of ACS diagnosis. ACS is a highly dynamic process that can produce subtle ECG changes. These changes may be nondiagnostic when viewed alone, but suggestive when viewed in temporal context. Unfortunately, because the standard ECG is insensitive and nonspecific for diagnosing ACS, the gains produced by serial assessment of standard 12-lead ECGs have been thus far been disappointingly small, even when highly trained observers do the ECG assessments.

Given the present scenario of approximately 4,000 sophisticated emergency departments distributed throughout the United States, this is a load of approximately 1,000 patients per year, per emergency department, that are undergoing significant testing for acute coronary syndrome, only to find out later that most of such patients had no cardiac problems. There is a need for easily adoptable and better tools to minimally invasively, and efficiently, determine which patients are indeed suffering from genuine acute coronary syndrome. While some products, such as the special vest-type apparatus available under the trade name PrimeECG from Heartscape Technologies, Inc., and the multi-electrode panel system described in U.S. Pat. No. 6,584,343 have attempted to address some of the shortfalls of conventional ECG analysis, they require suboptimal changes in the ECG data acquisition process, such as a requirement of a specific vest type apparatus intercoupled to a specific data acquisition system. It would be preferable to require a minimal amount of change to such processes in clinical environments such as the emergency room.

### BRIEF DESCRIPTION OF THE DRAWINGS

FIGS. 1A and 1B show the location of standard ECG leads on the body, and an example of a typical recording obtained therefrom.

FIGS. 2A-2C are block diagrams showing the collection and storage of ECG data, and its transmission, processing and analysis.

FIG. 3 shows a block diagram describing the steps of ECG data collection, processing, computation and analysis using the systems and methods described herein.

FIG. 4 shows a three-dimensional display of heart vector activity over time, using the systems and methods described herein.

FIG. 5 shows an example of a waveform from one of 12 standard ECG leads.

FIG. 6 shows a table comprising examples of parameters useful for cardiac diagnosis using the systems and methods described herein, and their analysis with multifactorial analysis protocols.

FIG. 7 shows an example of ECG waveform analysis using the systems and methods described herein.

FIG. 8 shows an example of mathematical analysis of ECG waveforms using the systems and methods described herein.

FIG. 9 shows an example of the steps that may be taken in multifactorial analysis of ECG waveforms using the systems and methods described herein.

FIG. 10 shows a block diagram describing the steps involved in one preferred embodiment for serial ECG analysis using the systems and methods described herein.

FIG. 11 shows a block diagram describing one approach to serial ECG analysis using support vector machines and the systems and methods described herein.

### DETAILED DESCRIPTION

The present inventors propose that serial (or dynamic) changes in electrocardiographic (ECG)-based markers can be used in the diagnosis of acute coronary syndromes (ACS), and can be used to differentiate ACS from a broad range of heart diseases, including but not limited to left ventricular hypertrophy (LVH), pericarditis, intraventricular conduction delays (IVCD), right bundle branch block (RBBB), benign early repolarization (BER), hypertrophic cardiomyopathies (HCM), dilated cardiomyopathies (DCM), infiltrative cardiomyopathies (ICM), and the like. The primary diagnostic problem created by such heart diseases is that they often produce ECG findings that may resemble the ECG changes produced by ACS. This creates the possibility of diagnostic delay, confusion, or outright error. Alternatively, pre-existing ECG changes related to such heart diseases may obscure important new ACS-related ECG changes, again making delay, confusion and error more likely. For example, patients with pre-existing LVH sometimes develop ACS, and the pre-existing ECG changes associated with LVH make it difficult to detect new ECG changes associated with the superimposed ACS.

In general terms, the present inventions incorporate serial electrocardiographic assessment with three-dimensional (3D) vectorial analysis of the cardiac electrical signal, using changes in novel 3D-based vectorial markers over time (i) to improve ACS detection (that is, to improve diagnostic sensitivity for ACS), and (ii) to improve differentiation of ACS from the broad range of heart diseases that may produce electrocardiographic changes that resemble ACS (that is, to improve diagnostic specificity for ACS).

### Static (Single ECG) Analysis of Novel ECG Markers, Including 3-D Based Markers, Substantially Improve Cardiac Diagnosis

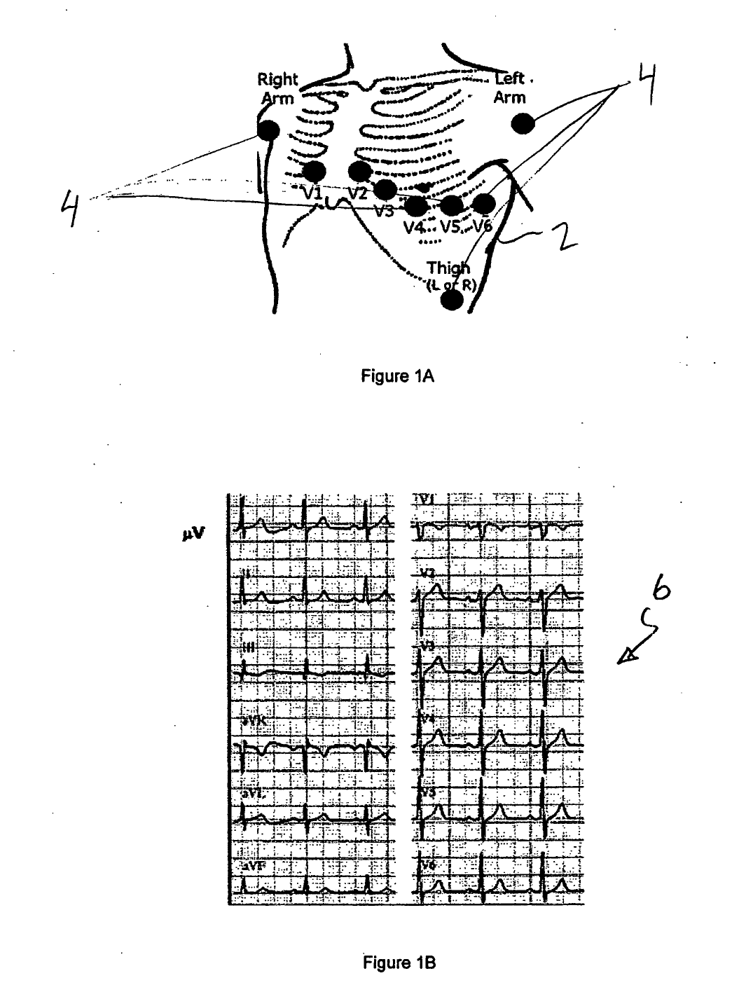

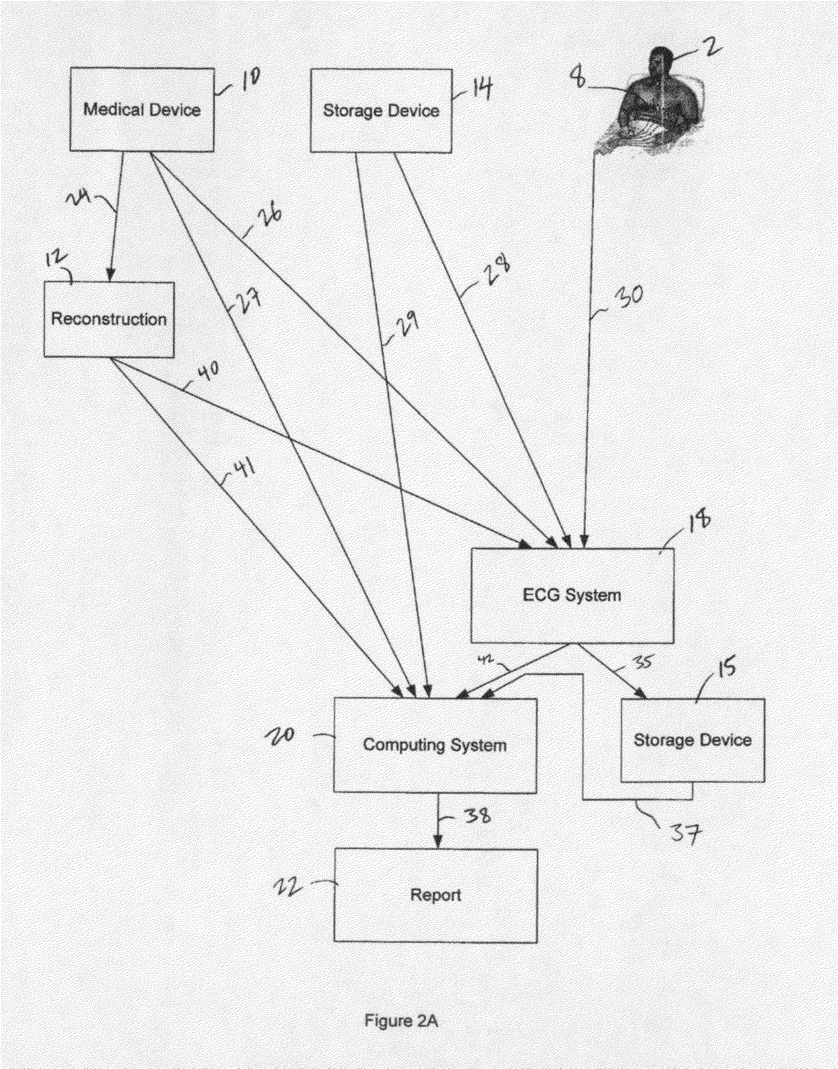

Referring to FIG. 1A, a typical ECG electrode location (4) configuration is depicted for capturing a standard 12-lead ECG from a patient (2). The data from the electrodes may be used with a conventional strip chart recorder or plotter to create an output (6) such as that depicted in FIG. 1B. As described above, aspects of this kind of conventional ECG output (6) are very useful in many types of diagnostics, and as it turns out, ECG data is rich with information beyond conventional ECG application, as described, for example, in U.S. patent application Ser. No. 12/484,156, entitled “Method for quantitative assessment of cardiac electrical events”, which is incorporated by reference herein in its entirety. To proceed with utilization of such data in further processing, an immediate challenge is capturing such data.

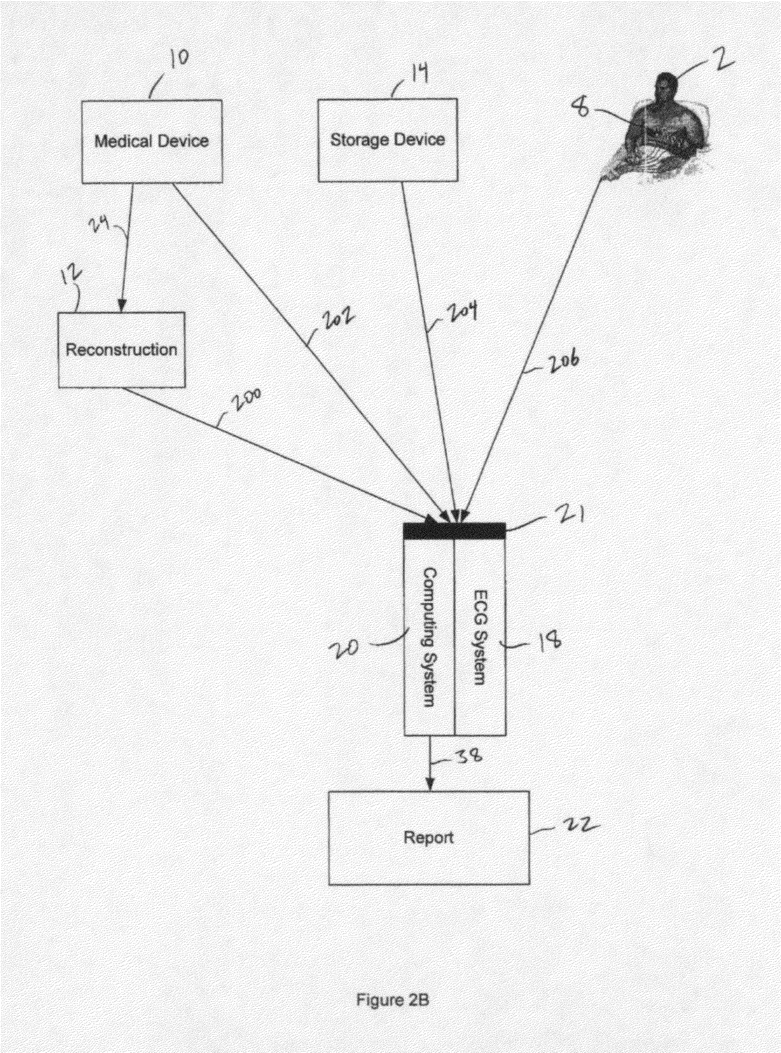

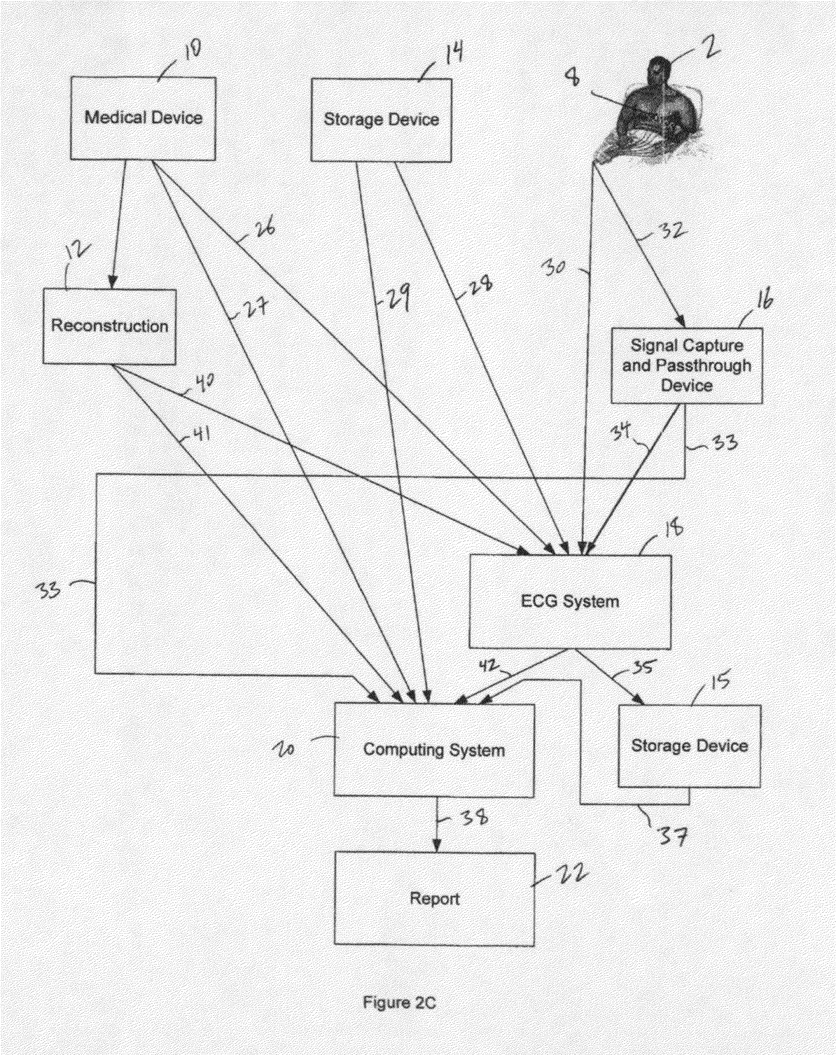

Referring to FIG. 2A, in one embodiment the data may be directed (30) from the patient (2) ECG electrodes (8) to an ECG system, such as those available from the Prucka Engineering division of GE Healthcare, Inc., and thereafter passed via a connection (42), in a form such as an electronic output file, to a computing system (20) configured to conduct detailed analysis of such data and ultimately facilitate production (38) of a report (22) configured to provide diagnostic information and/or conclusions to a healthcare operator. The ECG system (18) may be configured to filter, conduct an analog to digital conversion of, or store the pertinent ECG data before or after passing it to the computing system (20). The ECG system (18) may also be configured to pass (35) the data to a storage device (15) which may be used to provide the computing system (20) with access to such data through a connection (37) to the storage device (15), for example for a clinical scenario wherein a cardiologist wishes to review data and cases from an emergency department in an offline review scenario. Each of the connections between nodes, such as the patient, ECG system, computing system, storage device, and reporting mechanism, as well as other depicted devices, such as an additional storage device (14) and a medical device (10), may be conducted with a local wired connection, a local wireless connection, a remote wired connection, or a remote wireless connection, using modern information technology infrastructure. In another embodiment, such connection may be manually conducted by virtue of a memory device configured to be used to transiently move data from one node to the next. Data may be moved between devices in many ways, such as realtime, near-real-time, in transient packets, by manual storage devices transiently.

In another embodiment, the source of data may not be a live patient (2), but rather a device capable of providing ECG-related data which may be dispatched to other devices and/or stored upon memory which may be coupled to or reside within such device. For example, referring to FIG. 2A, in one embodiment, the source may comprise a storage device (14) such as a flash memory or hard disk drive, capable of storing significant amounts of information, and preferably connected (28, 29) via one of the aforementioned connection types to an ECG system (18) or other networked device such as a computing system (20). In another embodiment, the source of data may comprise a medical device (10), such as a Holter monitor, or a prosthesis, such as a defibrillator, pacemaker, or cardioverter which may or may not have a memory comprising stored ECG data, or stored reduced cardiographic vector set data, which may be used by a downstream computing device such as the ECG system (18) or computing system (20). Such device (10) may comprise a processor or microcontroller, and/or a memory device or interface. In one embodiment, the medical device (10) may comprise a product such as that available from NewCardio, Inc. under the tradename CardioBip®, described, for example, in U.S. patent application Ser. No. 10/568,868 by Bosko Bojovic, filed 2-21-06, incorporated by reference herein in its entirety; such device uses three non-standard ECG vectors and a reconstruction algorithm to produce a reconstructed 12-lead ECG recording from the three non-standard vectors. Preferably such device (10) is also connected (26, 27, 24) to other systems, such as the ECG system (18), computing system (20), or an intermediate computing device (12) configured for reconstructing a multi-lead ECG dataset, such as a 12-lead ECG dataset, from the reduced cardiographic vector set data which may be passed to it over a connection (40, 41). The intermediate computing device (12) may be incorporated or integrated into the medical device (10), the ECG system (18) or the computing system (20). Alternatively, its data may be stored in a storage device such as those illustrated as elements 14 and 15. Reconstruction of a multi-lead ECG dataset using reduced cardiographic vector set data from devices such as pacemakers or defibrillators has been discussed, for example, by Kachenoura et al in “Using Intracardiac Vectorcardiographic Loop for Surface ECG Synthesis”, EURASIP Journals on Advances in Signal Processing, Volume 2008, Article ID 410630, which is incorporated by reference herein in its entirety. Each of the connections referred to herein, such as those described above in reference to FIG. 2A (41, 40, 27, 26, 29, 28, 30, 42, 35, 38), may be configured as a local wired connection, a local wireless connection, a remote wired connection, or a remote wireless connection, using modern information technology infrastructure.

Referring to FIG. 2B, another embodiment similar to that depicted in FIG. 2A is depicted, with the exception that the embodiment of FIG. 2B features a computing system (20) that is closely integrated with the ECG system (18). In one embodiment, the computing system (20) may comprise a module housed within the housing of the ECG system (18). In another embodiment, the computing system (20) and ECG system (18) are directly coupled or directly physically integrated relative to each other. In another embodiment, the computing system (20) may comprise a removable module operatively coupled to the ECG system (18). Referring to FIG. 2B, an input interface (21) is depicted for capturing incoming connections and data. The integrated systems may share an integrated input interface (21), as illustrated in FIG. 2B, or may each have their own input interface. With a tight integration of the computing system (20) and ECG system (18), data may be shared and moved between both systems. A unified input interface (21) facilitates simplified connections (206, 204, 202, 200) with sources such as patient electrodes (8), storage devices (14), medical devices (10), and other devices such as an intermediate reconstruction device (12). In another embodiment, the ECG system (18) has its own front end interface to directly accept signals from ECG electrodes (8) and the computing system (20) has its own digital interface (e.g. USB port, Bluetooth, wired, wireless, etc.) to directly accept information and/or signals from a storage device (14), from a medical device (10), and/or from a 12-lead ECG reconstruction device or subsystem (12). In such preferred embodiment the interface (21) would be split, rather than unified as depicted in FIG. 2B, to address the needs of systems (18) and (20). In one embodiment, systems (18) and (20) and the elements of interface (21) are all coupled or physically integrated in the same housing.

Referring to FIG. 2C, another embodiment is depicted illustrating that ECG related data from any of the depicted sources (8, 10, 12, 14, 15, 16) may be processed by the computing system (20) in parallel to connectivity to the ECG system (18) given suitable connections (37, 29, 27, 41, 33) for the computing system (20) and suitable connections (34, 30, 28, 26, 40) for the ECG system (18); the ECG system (18) and computing system (20) may also be operably coupled (42) to share information; in another embodiment they remain independent and the direct connection (42) is nonexistent.

Referring again to FIG. 2C, in another embodiment, signals may be passed (32) from the patient (2) electrodes (8) to an intermediate device (16) configured to store and/or transmit (33, 34) such data to the computing system (20) or ECG system (18). The intermediate device (16) may, for example, comprise a mini-ECG system that provides 12-lead ECG data to the computing system (20). At the same time, intermediate device (16) may pass through the signals from patient electrodes (8) to a standard ECG system (18). In such system configuration, data connection (42) may not be necessary. The intermediate device (16) may also have a low-power flash memory device along with a transmission bus, such as a wired or wireless transceiver bus, configured to interface with the ECG system (18), computing system (20), or other connections or devices to which the ECG data may be directed. In one embodiment, the intermediate device (16) may comprise analog front-end electronics, protection networks (e.g. against defibrillation shocks, electrostatic discharges, etc.), amplifiers, a microcontroller or microprocessor capable of various levels of processing of the data, such as analog to digital conversion and/or digital or analog filtering of various configurations, before dispatch to other connected systems.

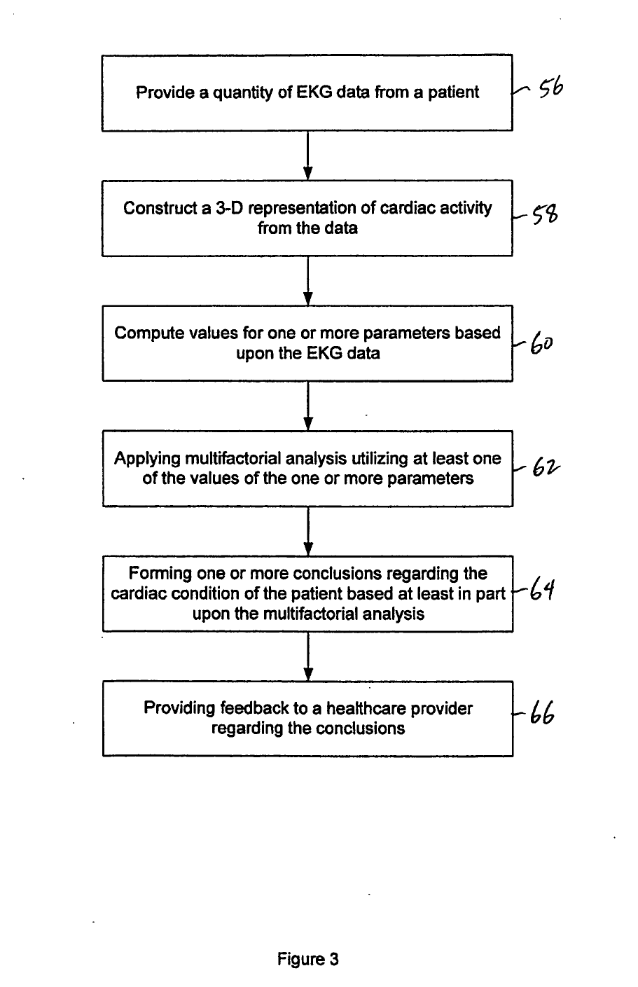

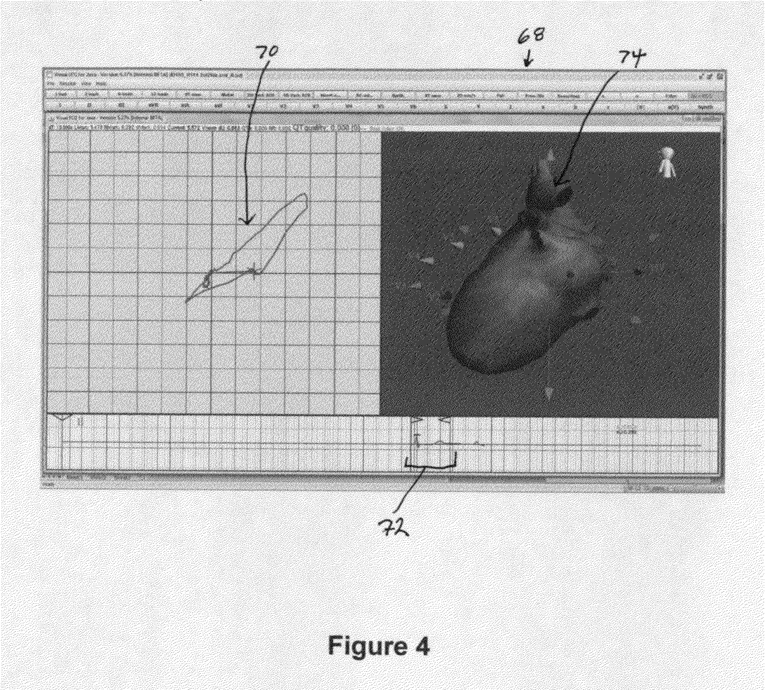

Referring to FIG. 3, systems such as those described in reference to FIGS. 2A-2C may be used to provide valuable feedback for healthcare providers (66). As shown in FIG. 3, a pertinent quantity of patient-related ECG data is provided (56). Using this data, a three-dimensional (“3-D”) representation of cardiac activity may be constructed from the data (58). Subsequently, values for one or more preselected parameters may be computed using the ECG data (60) (e.g. 3-D ECG data, 12-lead ECG data, or reconstructed 12-lead ECG). With such parameter values in hand, multifactorial analysis may be conducted (62) using at least one of the values of the one or more parameters, and, in accordance with a particular multifactorial analysis protocol, one or more conclusions regarding the cardiac first condition of the patient may be drawn (64), based at least in part upon the multifactorial parameter-based analysis. The step of creating a 3-D representation of cardiac activity may be conducted using 3-D vectorcardiography computer software on a computing system, such as those described in reference to FIGS. 2A-2C, with software such as that available from NewCardio, Inc., the assignee, and described at least in part in the aforementioned incorporated patent application. A typical 3-D representation of cardiac activity using such tools is depicted in FIG. 4, wherein the user interface (68) is configured to display a 3-D vector diagram (74), a plot (72) of a particular 12-lead trace portion being observed, and a loop diagram (70) pertinent to the portion. It is understood that such display is not required, or limiting, for the concept of 3-D representation of cardiac activity or for the operation of the invention. In one embodiment, referring to FIG. 3, the only visual output presented to the user (e.g. cardiologist, technician, emergency department doctor) may be in the form of a paper report coming out at step (66). A display, such as that illustrated in FIG. 4, may provide enhancing information, such as showing to the medical staff and estimated location of a cardiac infarct. The scope of this invention is not limited to visual or displayable types of 3-D representation of cardiac activity. Without limitation, computation of angles between QRS and T loops, for example, constitutes a 3-D representation of cardiac activity. Similarly, as in another example, computation of the magnitude of the cardiac vector constitutes a 3-D representation of cardiac activity. Yet as another example, conversion of a standard or reconstructed 12-lead ECG into X, Y, Z vectorcardiographic elements constitutes a 3-D representation of cardiac activity. Such transformation may be implemented, for example, as described by Dower in U.S. Pat. No. 4,850,370.

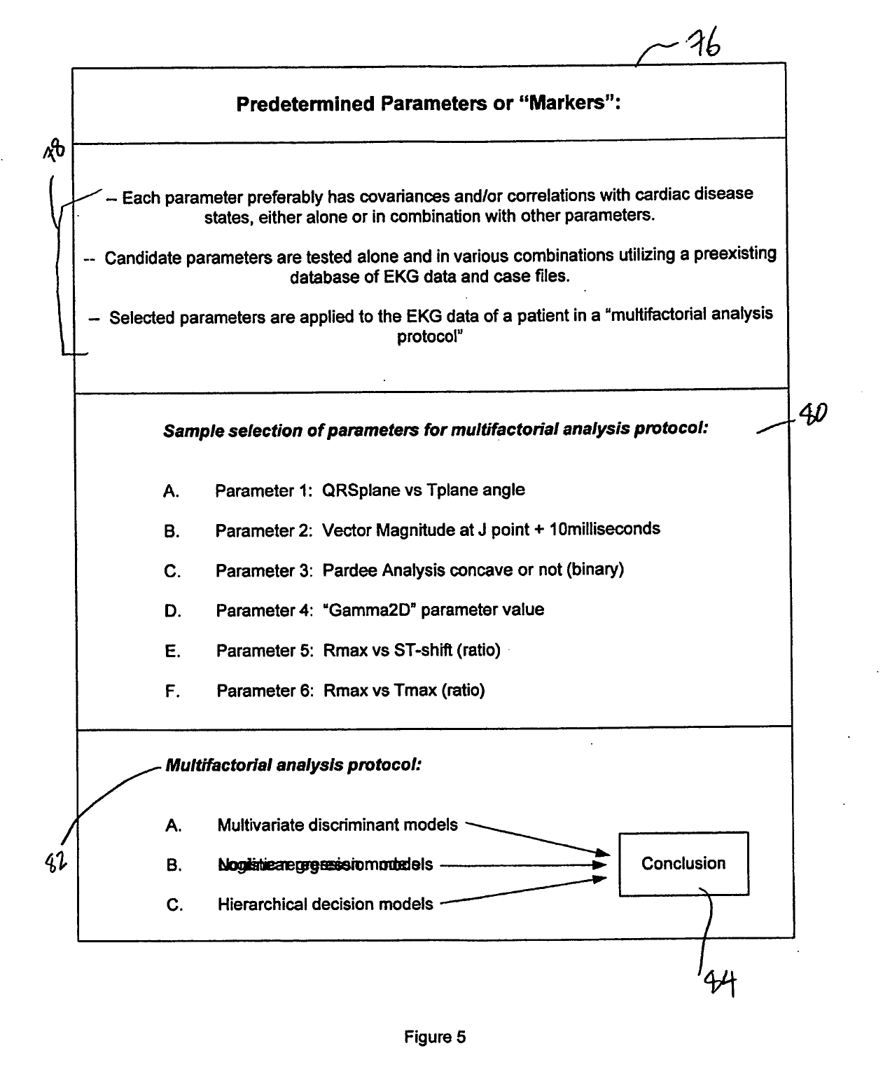

Referring to FIG. 5, the aforementioned predetermined parameters or “markers” preferably are selected for their ability to assist in the clinical diagnosis of patients in a particular group, versus patients not in such group. As shown in the table (76) of FIG. 5, each selected parameter preferably has several characteristics (78). Referring to FIG. 5, covariances and/or correlations with cardiac disease states, such as acute coronary syndrome, either alone, or in combination with other parameters are preferred; further, candidate parameters preferably are tested alone and in various combinations/permutations using a preexisting database of ECG data and case files to determine which combinations and/or permutations have the best resolution in terms of the desired results. After such preferred combinations and/or permutations have been determined, they may be used by a computing system and applied to the ECG-related data of a particular patient in a multifactorial analysis protocol wherein more than one parameter-based sub-analysis is combined to create a decision analysis conclusion.

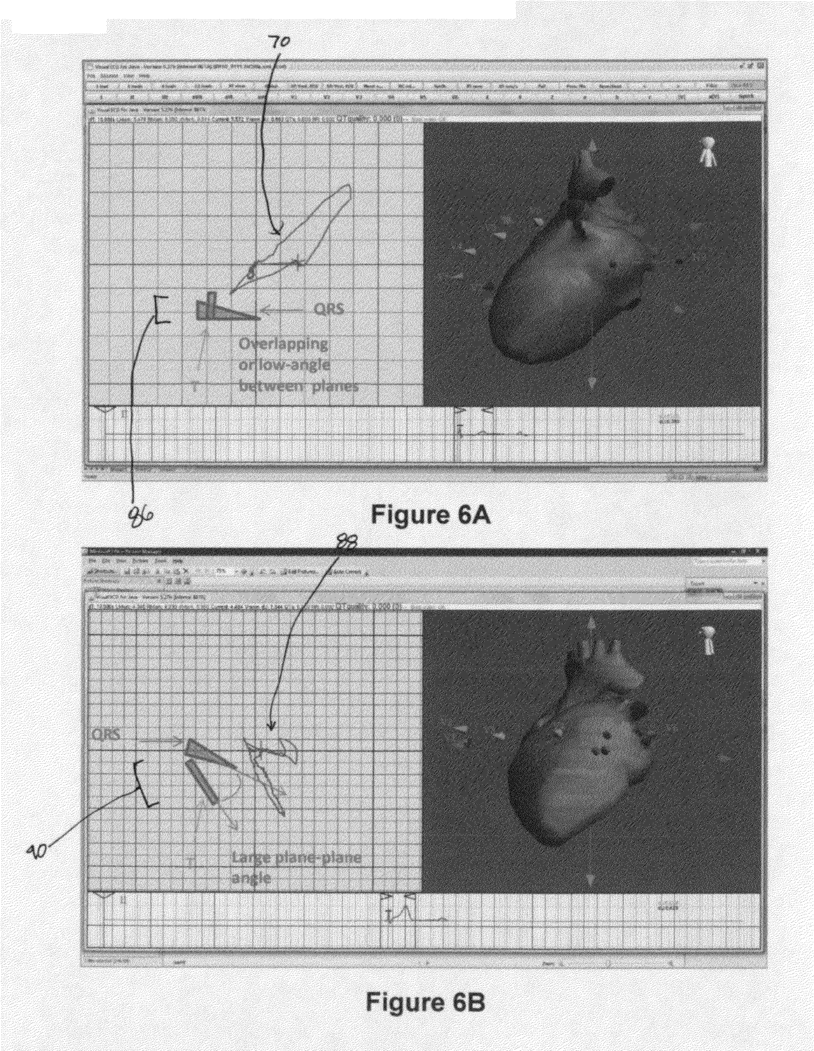

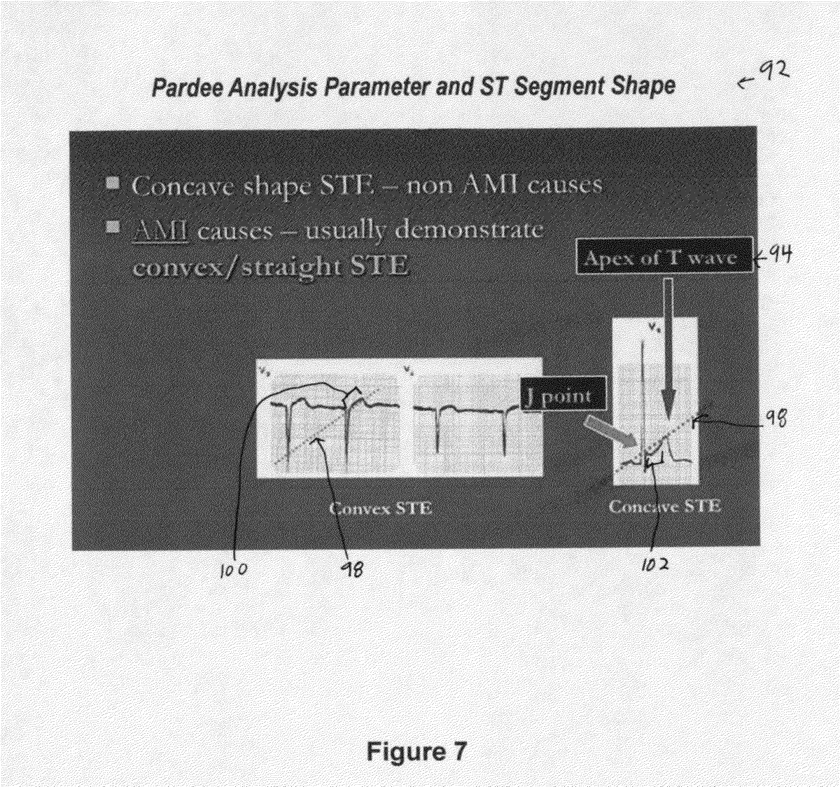

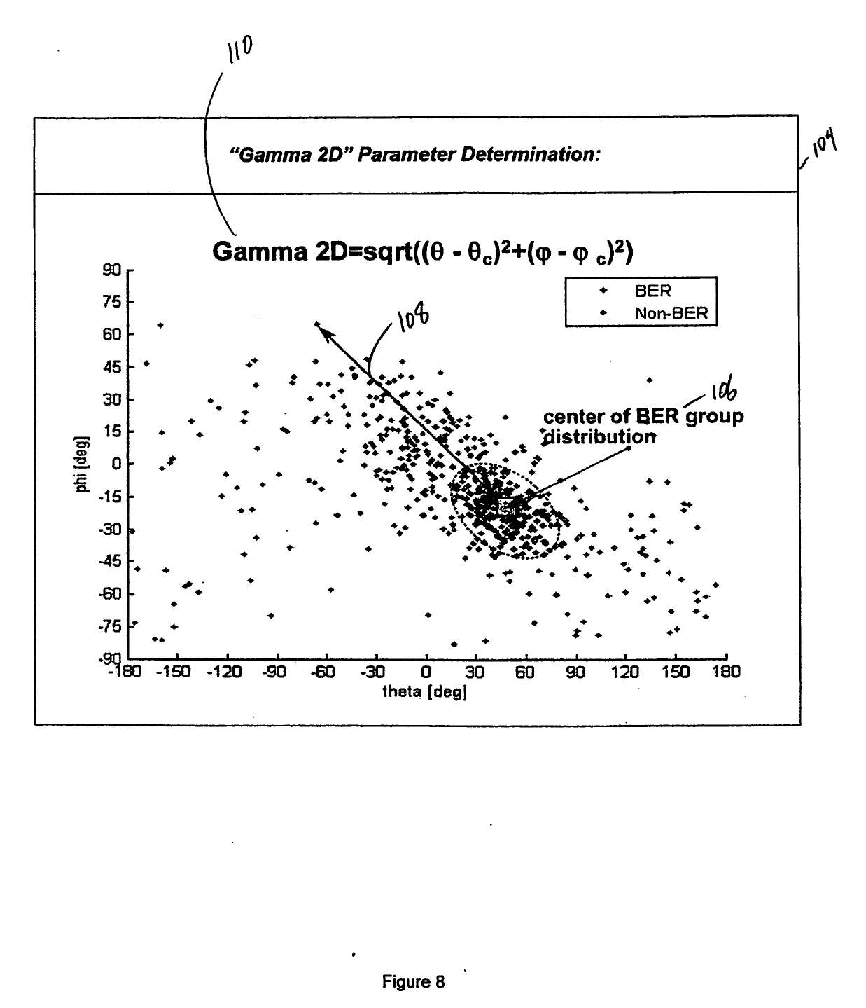

We have found in our experimentation that many candidate parameters or markers are useful in conducting cardiac ECG diagnostic analysis. For example, referring to FIG. 5, a listing (80) of a few is depicted, including a ratio of QRS plane angle versus Tplane angle, as described further in reference to FIGS. 6A and 6b, the QRS plane and Tplane angles being available from 3-D cardiography analysis; the vector magnitude from 3-D cardiography analysis at a point 10 milliseconds after the J-point on ECG; a determination (binary) of Pardee type concavity or not, from either the ECG data or the 3-D cardiography analysis, as described further in reference to FIG. 7; a “Gamma 2D” parameter value, as described further in reference to FIG. 8; and ratio-metric parameters such as the ratio of Rmax versus ST-shift from the ECG data, or the ratio of Rmax versus Tmax from the ECG data. The term Rmax refers to the peak of an R wave computed on the 3-D ECG representation (e.g., on the magnitude of the cardiac 3-D vector); the term ST-shift refers to the shift seen in the ST segment of the ECG vector magnitude; the term Tmax refers to the peak of the T wave of the ECG vector magnitude. Referring again to the table (76) in FIG. 5, a multifactorial analysis protocol (82) may comprise multivariate discriminant models, regression models, support vector machine models, and/or hierarchical decision models, to employ the various parameter values in furtherance of a clinically impactful conclusion (84). Further, in one embodiment, one or more confidence indices are computed regarding one or more of the conclusions based at least in part upon the one or more parameter values, preferably using further numerical analysis. For example, female patients younger than 65 years of age that present to emergency departments with non ST elevated myocardial infarction (NSTEMI) typically present confounding ECG morphologies. In one embodiment, applying multifactorial analysis to process data from such a patient, a myocardial infarction detection may be hypothetically rendered, and such conclusion may have a lower than average probability of being correct due to the confounding issues. One embodiment may be configured to use a self-computed confidence threshold that estimates the chances of its output being correct. If the chances of providing a correct detection output fall below this threshold, then the system may be configured to advise the healthcare provider of the detection result and of the decreased confidence level. In one embodiment, parameters in multifactorial analysis may be selected based upon a factor such as patient age, gender, race, residency, citizenship, occupation, or profession.

Referring to FIGS. 6A and 6B, loop plots may be used as parameters or elements thereof. For example, the angle between the QRS plane (or any subplane, for example just the subplane corresponding to the QR or RS portion, or a subplane corresponding to any part of the QR or RS portion) and T plane, different between the two specimens depicted in FIGS. 6A and 6B, may be used as a parameter. In non-ACS patients, it is expected that the QRS plane (or any subplane) and the T plane form a relatively low angle. This angle has been observed to increase in patients with ACS. In one embodiment, a threshold in the range of 20°-40° may be used to separate ACS from non-ACS patients. In addition to loop plane angulation, loop planarity (i.e., how planar is the loop), and loop shape, such as circularity or correspondence with an elliptical shape (i.e., how close is the loop to the shape of a circle or ellipse), may be used as parameters. For example, non-ACS patients tend to have QRS and T loops that are close to planar. Conversely, ACS patients tend to have QRS and T loops with geometric deviations from planar figures. To establish planarity, a summation of unsigned distances of points on the loop with respect to a reference plane, such as the principal component analysis plane, may be used as a planarity index. The lower the sum, the more planar the loop would be. As shown in FIG. 6A, the QRS loop (70) is approximately planar. The depicted loops were constructed from non-ACS ECG data, based on the process described in reference to FIG. 3. FIG. 6B illustrates a QRS loop (88) that cannot be reasonably approximated as planar. The loops in FIG. 6B were constructed from ECG data associated with an ACS patient, based also upon the process described in reference to FIG. 3. Also illustrated in FIGS. 6A and 6B, 428 the QRS-T angle (86 and 90, respectively) has a relatively low value for the non-ACS data, and a relatively high value for the ACS data, respectively. Thus one of the one or more parameters used in multifactorial analysis may be based upon the planarity of one or more vector cardiogram loops relative to a reference plane, where the loops are any of the P, QRS, or T loops, or segments thereof, as computed in the 3-D representation of the ECG data.

Referring to FIG. 7, ECG signal analysis known as Pardee analysis, named after Harold Pardee's research in the 1920's, may be used to generate a parameter. In essence, if the a line (98) drawn between the J point (96) and the apex of the T wave (94) shows a convex or straight ST signal (100), the patient is more likely to have a myocardial ischemia or infarction that is a patient with a concave ST signal (102) in the same location, and thus this Pardee parameter is useful in clinical diagnosis of ACS.

Referring to FIG. 8, we have created a parameter we refer to as “Gamma 2D”, which we find to be clinically valuable.

Benign early repolarization (“BER”) is a condition that a particular patient will either have or not have. It is also one of the most frequent confounders of 12-lead ECG analysis that causes false positive diagnoses of ACS in clinical settings. We have found that the theta and phi (the angular coordinates of the ST vector) are very tightly clustered for a BER patient group, and very distributed for non-BER patients. Thus, we find the Gamma 2D marker, which is the position of the ST vector (106) relative to the center of the early repolarization distribution (106), to be a useful parameter. In another variation, the Gamma 2D marker may be defined as the position of the T vector (not shown) relative to the center of the early repolarization distribution. For clarity of terminology, a first cardiac condition will be used in reference to a cardiac condition that a clinician is trying to detect, while a second cardiac condition will be used in reference to a confounding condition (for example, BER, LVH and RBBB are three particular confounding second conditions that may be of interest). An objective is to eliminate the confounding problem to improve the performance of detection of the first condition. In some variations, other second conditions such as left ventricular hypertrophy (LVH) or right bundle branch block (RBBB) may be used to establish the centerpoint of the distribution. The ST vector is a vector constructed based on the orientation of the cardiac vector at points such as the J point, J point+40 milliseconds (ms), J point+60 ms, J point+80 ms, or J point+another temporal amount that shifts the cardiac vector towards the peak of the T wave (the “T point”), all such points represented on the 3-D representation of the ECG data. The T vector is the cardiac vector at the peak of the T wave. Alternatively, the cardiac vector orientation at the end of the T wave could be used to represent the T vector.

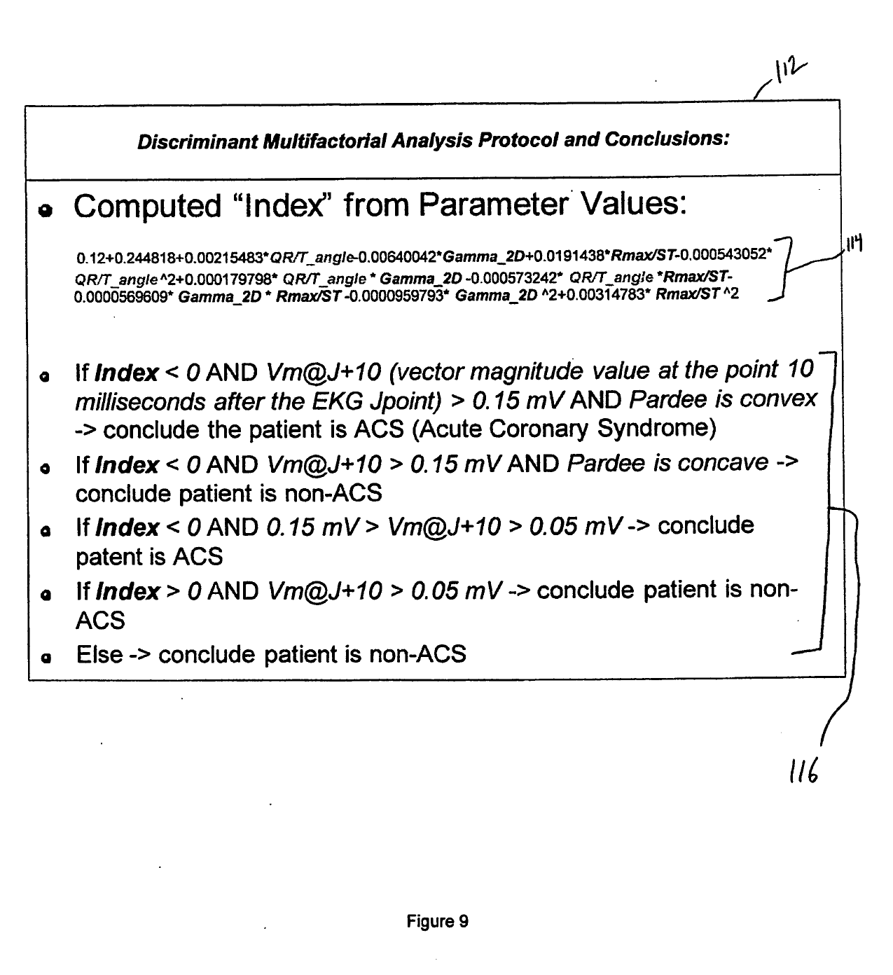

Referring to FIG. 9, one preferred embodiment of a discriminant multifactorial analysis protocol (112) is depicted, wherein calculation of a numerical “Index” based upon various parameters (QR/T angle; Gamma 2D; Rmax/ST ratio—element 114 is the equation for Index) and a series (116) of discriminant tests leads to clinical conclusions. The Index and the diagrammatic flowchart in FIG. 9 showed substantial improvement in the detection of ACS in a study performed on 460 all-corners patients that reported to an emergency department with angina. By additional clinical test (e.g. troponin tests); only 140 of these patients were confirmed to have had ACS. The algorithm represented in Figure resulted in a sensitivity of 78% and specificity of 84%. The same patients were reviewed by two expert certified, practicing cardiologists using only 12-lead ECG data. Their readings provided an averaged sensitivity of only 57% and an averaged specificity of 89%. Therefore, the algorithm improved by more than 20% the expert human reader sensitivity in detecting ACS while preserving the specificity at equivalent levels.

### Dynamic (Serial) Analysis of ECG Markers for Cardiac Diagnosis, Including 3-D Based Markers

Serial analysis of novel 3D-based vectorial markers. A detailed description of 3D-based vectorial markers, and how they are generated from a body-surface electrocardiographic recording, is available in [my3KG patents and applications], which are incorporated herein by reference in their entirety, and are also summarized in the present patent application, particularly in FIGS. 3-9 and accompanying text. In accordance with the inventions disclosed and described herein, 3D-based vectorial markers may be very broadly classified as (i) vector magnitude (VM) signal markers, (ii) 3D markers, and (iii) markers based on a degree of variability of certain ECG parameters.

Examples of vector magnitude (VM) signal markers include without limitation (i) time duration markers, e.g., based on a duration of a specified portion of the RR interval or a ratio of the durations of two different specified portions of the RR interval, (ii) voltage markers such as a measured voltage at a particular time point on the RR interval or a ratio of the measured voltages at two defined time points of the RR interval, or (iii) combined time-voltage markers, such as a two-dimensional area covering some portion of the VM signal, a Twave slope marker, or a QRS wave slope marker.

Examples of 3D markers include without limitation (i) T-loop markers, such as Tvelocity markers, Tangle markers, and markers based on the morphology of the T-loop (planarity, roundness, symmetry, etc.), (ii) QRS loop markers, such as QRS velocity markers, QRS angle markers, and markers based on the morphology of the QRS-loop (planarity, roundness, symmetry, etc.), or (iii) combined QRS-T-loop markers, such as angles between directions of QRS and T loop, and angles between QRS and T loop planes.

Examples markers based on the degree of variability of some ECG parameters include without limitation (i) markers based on a variability of the parameters defined on the VM signal, and (ii) markers based on a variability of the parameters defined on the respective T-loops and QRS loops.

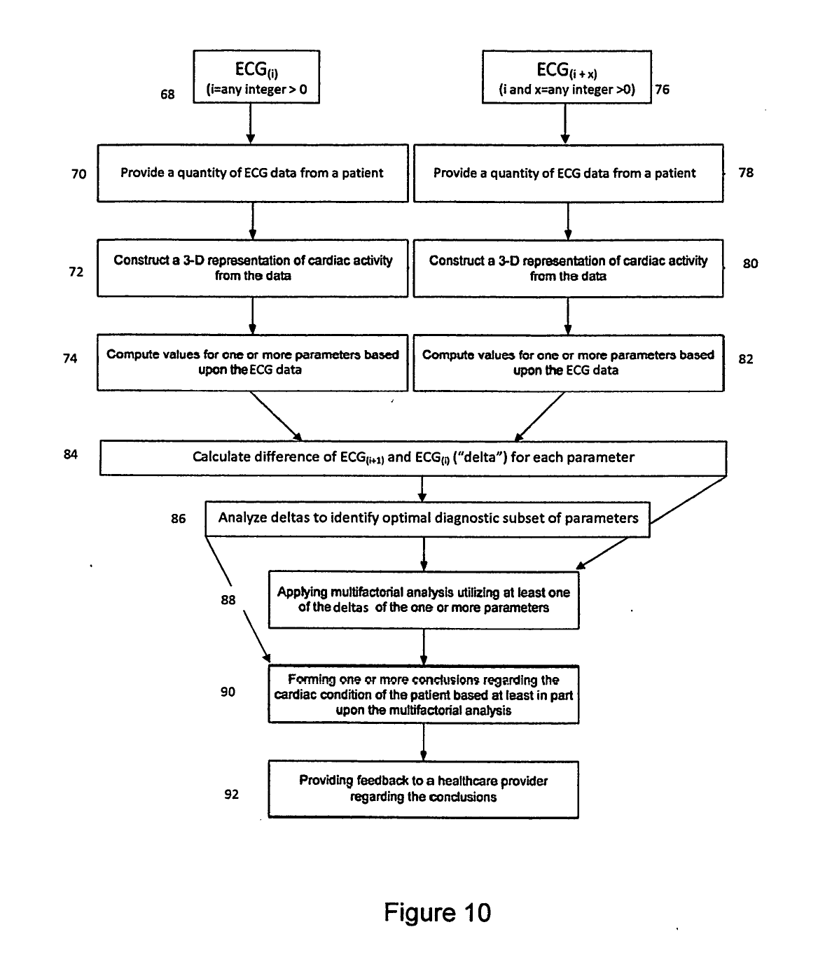

Referring to FIG. 10, one preferred embodiment for serial ECG analysis is depicted. For serial analysis, two or more ECGs obtained at different times are obtained for an individual patient (for example, FIG. 10. depicts serial comparison of ECG₍ᵢ₎ (68) and ECG₍ᵢ₊ₓ₎ (76), where i and x are any integer greater than 0). The difference in time between the two or more ECGs undergoing serial analysis can be any amount. For example, in current medical practice, many ECGs are recorded for a few seconds, or about 6 seconds, or about 10 seconds, whereas others may be continuously recorded for extended periods, such as 12 hours, 24 hours, 48 hours, one week or longer. Regardless of their duration, for serial ECG analysis the second ECG in the series may be initiated instantaneously after the first is completed (or may even be partially overlapping with the first), or it may be separated by one second, or a few seconds, or 10 seconds, or 1 minute, or 5 minutes, or 10 minutes, or 15 minutes, or 30 minutes, or longer, such as 1 hour, 2 hours, 3 hours, 4 hours, 8 hours, 24 hours or even longer—so long as they are not exactly simultaneous. In the case of ECGs recorded continuously over extended periods (for example, Holter monitors and the like), segments of the recording of any length may be compared by serial analysis to other segments obtained at a different time in the recording, so long as the two or more compared segments are not completely overlapping. When more than two ECGs are compared by serial analysis, the time interval may be constant, or may be highly variable, without material impact on the analysis. In addition, serial analysis is indifferent to the source of the ECG data; for example, the first ECG may be obtained from a standard 10-sec ECG obtained at the patient's bedside, while the second, third, fourth, or greater ECGs may be obtained from Holter recordings, CardioBip recordings, or any other suitable method for obtaining ECG data.

Referring again to FIG. 10, for serial ECG analysis a quantity of ECG data for a patient is provided for at least two non-simultaneous ECGs (70, 78), in the same manner described in FIG. 3 and accompanying text in the present patent application. The ECGs undergoing serial analysis may obtained in any of the various manners described in the preceding paragraph and elsewhere in this application. A 3-D representation of cardiac activity (72, 80), and values for one or more parameters (82, 84), are then computed for each ECG undergoing serial analysis, as described in FIG. 3 and accompanying text. For each computed parameter, the difference (“delta”) is calculated for that parameter (84) between the two or more ECGs undergoing serial analysis.

Referring again to FIG. 10, after calculation of deltas for each parameter, it is possible to proceed directly to multifactorial analysis, using at least one of the deltas for one or more parameters (88). Multifactorial analysis is done in the manner described in FIG. 3 of this application and accompanying text.

In a preferred embodiment, an initial analysis of the deltas (86) is done prior to multifactorial analysis. The initial analysis may be done using statistical methods that are well known in the art. For example, a subset of the deltas calculated for each parameter may be analyzed by multivariable analysis, multisample inference, analysis of variance, and/or analysis of covariance, techniques for which are described in standard textbooks of biostatistics and clinical research. See, e.g., Bernard Rosner, Fundamentals of Biostatistics, 7^{th} Edition 2011, incorporated by reference in its entirety. The subset may be comprised of deltas for a single subject, or in a preferred embodiment, the subset may be comprised of mean delta values across more than one subject, or mean of the absolute delta value across more than one subject. It is important for optimal diagnostic accuracy of serial ECG analysis to determine if a particular parameter is stable or unstable over time, and the use of absolute delta values allows detection of instability in the circumstance where the parameter is unstable but moves in different directions in different subjects. Several widely available software packages well known in the art are available to perform statistical calculations, for example SAS, SPSS, JMP, MINITAB, Excel, and the like. In this manner, an optimal subset of calculated deltas may be identified that have the highest sensitivity, specificity or predictive value for cardiac diagnosis, for example diagnosis of AMI.

When multiple ECGs (3 or more) are available for serial analysis, additional statistical calculations may further increase accuracy for cardiac diagnosis. Multiple ECGs may be available from any source, for example multiple ECG recordings from whatever source (e.g., standard bedside 12-lead ECG, recordings from a CardioBip device) taken from patient over time, or serial ECGs extracted from continuous monitors such as multiple lead Holter recordings. When multiple ECGs are available, statistical analysis for any marker can include descriptive statistics such as mean delta value, standard deviation, standard error, confidence intervals and the like. For example, when the specified confidence interval (e.g., 90% or 95%) around the mean delta value excludes 0, it indicates that the marker is varying significantly over the time interval being studied. The mean delta for any parameter may be calculated as the true mean, or as the mean of the absolute values of the delta differences. By using absolute values of deltas rather than true delta value, it is easier to detect delta instability across multiple subjects in the instance where a marker may change in one direction in some subjects, and the opposite direction in others. In such a circumstance, the mean of absolute delta values will be large and indicate instability for a parameter, even though the mean of the true delta values may be small and misleadingly suggest stability for that marker. Since AMI and other forms of acute coronary syndrome are highly unstable (time-variable), whereas other cardiac conditions tend to be stable (less time-variable), a confidence interval around a mean delta value or mean delta absolute value that excludes 0 suggests that the patient has AMI or other form of acute coronary syndrome, whereas a confidence interval that includes 0 suggests that the delta is more stable and AMI or acute coronary syndrome is less likely. One skilled in the art having the benefit of this disclosure can readily see that once this analysis is completed, one may then proceed to higher-level statistical analysis, such as multivariable analysis, multisample inference, analysis of variance, and/or analysis of covariance, as described in Rosner (op. cit.) and the discussion above. The results of this statistical analysis may form the basis of conclusions regarding the cardiac condition of the patient (90), or may form the basis of additional multifactorial analysis (88) done prior to generating conclusions.

### Example 1

Distinguishing AMI from non-AMI using Serial ECG analysis. A total of 201 pts, 65.25% male, 57.2±13.2 yrs, experienced chest pain and presented to an urban ED (113 pts) or to a cardiac catheterization laboratory (CL) (88 pts). Of these, 112 pts had a final clinical diagnosis of AMI (52 STEMI, 60 NSTEMI) and 89 pts had no AMI. STEMI stands for ST Elevated Myocardial Infarction, whereas NSTEMI stands for Non-ST Elevated Myocardial Infarction. The medical records obtained at discharge from the ED or CL were used to establish our AMI/nonAMI gold standard. Two ECGs were taken for each patient between 10-60 min apart, and were transformed to 3D ECGs as described. Parameters, such as QRS-T angles, planarity of QRS and T loops, directional changes in the ST vector and ratio-metric markers, such the relative change in the peak of the R wave with respect to the shift in the ST segment, as measured on the vector magnitude (VM) ECG, were extracted and constituted our set of 3D ECG markers. A total of 41 3D ECG markers were evaluated.

In this example, mean of the absolute value of the deltas were calculated for each of the 41 parameters from the 201 subjects. The mean absolute delta was compared across two groups: those with a clinical diagnosis of AMI (112 subjects) and those with a clinical diagnosis of no AMI (89 subjects). The mean absolute delta value was greater in the AMI group than in the non-AMI group for 21 of 41 parameters (51.2%), and the average mean absolute delta value was 16.8% higher in the AMI group relative to the non-AMI group. From this group, we identified 6 parameters where the mean absolute delta value was markedly higher (>50%) in the AMI group. Of these, the largest difference (322% increase for AMI over non-AMI) was observed for the gamma 2D parameter described herein in FIG. 8 and accompanying text. Thus, the gamma 2D is highly useful for AMI diagnosis in a single ECG, but it becomes markedly more powerful for AMI diagnostic purposes when serial ECG analysis is used.

### Example 2

### Application of Genetic Algorithms and Support Vector Machines to Serial ECG Analysis

The diagnostic effectiveness of the ECG can be augmented by 3-dimensional (3D) vector analysis [4]. 3D ECGs provide additional information that may improve diagnostic accuracy [4-5]. Along with a 3D approach, the use of information from consecutive or serial ECGs (SECG) has been shown to increase sensitivity in the diagnosis of Acute Myocardial Infarction (AMI) (M. Salerno, P. C. Alguire, H. S. Waxman, “Competency in interpretation of 12 lead electrocardiograms: a summary and appraisal of the published evidence,” Annals of Internal Medicine (2003) 138:751-759). However, the aforementioned study focused only on two-dimensional ECG markers, particularly ST segment instability; we hypothesize that instability in 3D ECG markers would improve AMI diagnosis. Such 3D markers include, for example, angular, temporal, planarity, and ratio-metric parameters, as discussed earlier in this patent application.

To test the diagnostic capability of SECG analysis of 3D markers, we extracted 3D ECG markers from a set of 201 patients (pts) who had presented to a hospital emergency department (ED) with symptoms of chest pain. The final clinical diagnosis of AMI (acute myocardial infarction) or non-AMI, as provided by the full medical records, constituted the “gold standard” against which SECG analysis was compared. The changes (“deltas”) in 3D ECG markers, as extracted from SECGs, were processed using support vector machines (SVM), which have been shown to be useful for diagnosing heart disease using the standard 2-D ECG (A. E. Zadeh, A. Khazaee, V. Ranaee. “Classification of the electrocardiogram signals using supervised classifiers and efficient features”, Computer Methods and Programs in Biomedicine (2010) 99:179-194). By constructing an optimal separating hyperplane using the maximum margin between data points belonging to different classes, the SVM provides a reliable binary classification in a high dimensional feature space.

To optimize the training data and feature space, we utilized a genetic algorithm search (GA). The GA is an evolutionary algorithm search that operates on the principles of Darwinian evolution (Said Y H, “On Genetic Algorithms and their Applications”, Handbook of Statistics (2005) 24: 359-390). In the present study, the classification error rate was minimized with respect to a known subset of patients.

We present a multilayer of support vector machines with features, training data, and parameters optimized with genetic algorithms (GA-MLSVM) aimed at improved AMI detection accuracy. Our approach shows substantial sensitivity gains and relatively equal specificity compared to average cardiologists' diagnosis.

A total of 201 pts, 65.25% male, 57.2±13.2 yrs, experienced chest pain and presented to an urban ED (113 pts) or to a cardiac catheterization laboratory (CL) (88 pts). Of these, 112 pts had a final clinical diagnosis of AMI (52 STEMI, 60 NSTEMI) and 89 pts had no AMI. STEMI stands for ST Elevated Myocardial Infarction, whereas NSTEMI stands for Non-ST Elevated Myocardial Infarction. The medical records obtained at discharge from the ED or CL were used to establish our AMI/nonAMI gold standard. Two ECGs were taken for each patient between 10-60 min apart, and were transformed to 3D ECGs [4]. Parameters, such as QRS-T angles, planarity of QRS and T loops, directional changes in the ST vector and ratio-metric markers, such the relative change in the peak of the R wave with respect to the shift in the ST segment, as measured on the vector magnitude (VM) ECG, were extracted and constituted our set of 3D ECG markers. Percent changes in 3D ECG marker values across each patient's SECG were also computed. Initially, a total of 227 3D ECG markers were extracted.

Genetic algorithms are a set of evolutionary algorithms that operate on the principles of natural selection: mutation, selection, crossover, and reproduction. Said YH, “On Genetic Algorithms and their Applications”, Handbook of Statistics (2005) 24: 359-390, incorporated by reference herein in its entirety. A number of potential solutions to minimization problems are evaluated using a user defined fitness function. These solutions undergo the aforementioned principles and reproduce for new, fitter generations. The process repeats until the change in an error function ceases to exceed a specified value.

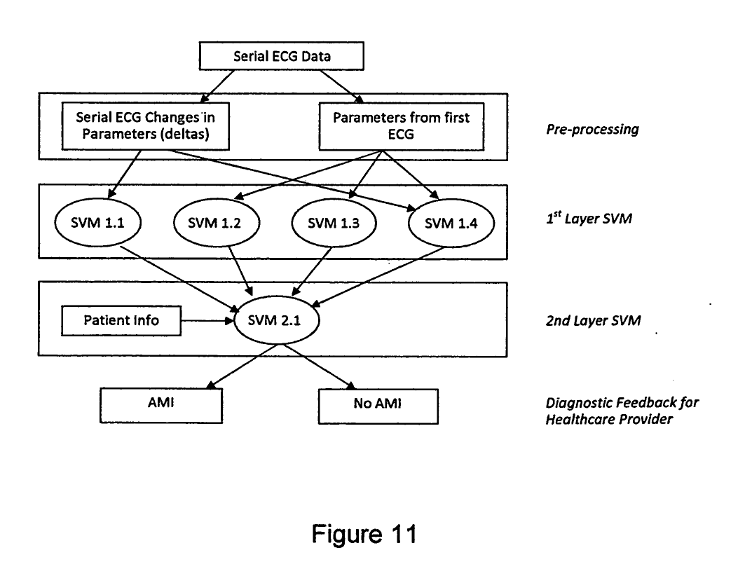

The selection of features and training data were optimized so to minimize the error rate of specificity and sensitivity for the network. Features were reduced from 227 to 60 as their fitness was determined from classification error using the generalized multilayered support vector machine (MLSVM; shown schematically in FIG. 11 and discussed in accompanying text) on all patients. Following feature reduction, training patients were chosen by using the same fitness function with an additional constraint on the number of training patients to be less than 100. These patients constituted a training set for which the SVM could be most generalized.

Multilayered Support Vector Machine. Let xεRⁿ denote a set of features, our 3D ECG markers, to be classified into y=±1. Let {(xᵢ,yᵢ), i=1, 2, . . . , l} denote a set of l training examples [3].

In the case of non-linearly separable data, the SVM finds a linear decision function f(x) that maps x to some higher dimension space where f(xᵢ)≧0 for y=+1 and f(xᵢ)≦0 for y=−1 [3]. Function f(x) provides a hyperplane that can be found by maximizing the margin between borderline points of separate classes [3].

Support vector machines were used in a multilayer network to classify each patient as AMI or non-AMI based on the computed features and changes in features. Referring to FIG. 11, a block diagram is presented, in which preprocessing, the 1^{st} layer SVM, and the 2^{nd} layer SVM are shown.

A radial basis function (RBF) kernel was chosen for all SVM with σ=15 and C=1. The 1^{st} layer SVM consisted of multiple SVM modules that simultaneously analyzed changes or deltas in 3D ECG markers from SECGs as well as the marker values from the patient's first ECG. Each SVM in this layer was trained on a subset of the patient data. SVM 1.1 was trained on SECG changes from subset A (30 ED pts, 50% AMI). SVM 1.2 was trained on 3D ECG marker values from the subset A. SVM 1.3 was trained on 3D ECG marker values from subset B (30 CL pts, 50% AMI). SVM 1.4 was trained on SECG changes and 3D ECG marker values from subset C (30 pts, 50% NSTEMI, 50% non-NSTEMI) from all 201 pts.

The binary outputs of the 1^{st} layer became features for the 2^{nd} layer. The 2^{nd} layer consisted of a single SVM that integrated 1^{st} layer outputs with higher order characterizations of the patients to give a final classification of AMI or non-AMI. SVM 2.1 was trained on subset D (24 pts, 50% AMI) based on the aforementioned features. In total, 70 patients were used for training due to the overlap between subsets A, B, C, and D.

The GA-MLSVM algorithm was tested on all 201 pts, all non-train pts (131 pts), and 1000 random subsets of all 201 pts consisting of the following: 20 STEMI, 20 NSTEMI, and 60 Non-MI pts. Additionally, blind testing was performed on a set of 12 pseudo-ischemia pts. They had been previously diagnosed with Benign Early Repolarization (BER), a condition that displays ST segment elevation but no AMI.

As shown in the table below, on all 201 pts, GA-MLSVM attained a sensitivity of 86.61%, a specificity of 91.01%, a positive predictive value (PPV) of 92.38%, and a negative predictive value (NPV) of 84.38%. On the 131 non-train pts, GA-MLSVM attained a sensitivity of 85.71%, a specificity of 88.33%, a PPV of 89.55%, and a NPV of 84.13%. FIG. 2 presents the mean, min, and max values for the aforementioned metrics computed on the randomized subsets. Additionally, the sensitivity of the algorithm on STEMI and NSTEMI patients are reported as 90.47%±5.08% and 83.18%±7.01% respectively. Since, on average, cardiologists exhibit 51% sensitivity and 91% specificity in AMI detection based on first collected ECG of ED patients complaining of chest pain, the mean performance of the SECG based GA-MLSVM improved sensitivity by 35.82% and had a negligible effect on specificity [4]. Finally, 11 out of 12 pseudo-ischemia pts were correctly classified as non AMI, for a specificity of 91.67%.

| Metric | Mean +/− St. Dev | Min | Max |
|---|---|---|---|
| Sensitivity | 86.82% +/− 4.23% | 75.00% | 100.00% |
| STEMI | 90.47% +/− 5.08% | 75.00% | 100.00% |
| NSTEMI | 83.18% +/− 7.01% | 60.00% | 100.00% |
| Specificity | 91.05% +/− 2.10% | 86.67% | 98.33% |
| PPV | 86.67% +/− 2.79% | 80.00% | 97.30% |
| NPV | 91.27% +/− 2.58% | 84.13% | 100.00% |

GA-MLSVM performed strongly, as exhibited by the highly improved sensitivity as compared to cardiologists' average. The excellent performance on various metrics demonstrates two points: the viability of using SECGs as classification features and the robustness of GA-MLSVM as a diagnostic tool for AMI detection. The high performance on the blinded pseudo-ischemia set indicates that the algorithm is not fooled by 2-D ST segment instability in non AMI patients. The combination of GA-MLSVM with analysis of SECGs improves diagnostic accuracy of AMI and non-AMI patients.

While multiple embodiments and variations of the many aspects of the invention have been disclosed and described herein, such disclosure is provided for purposes of illustration 25□ only. For example, wherein methods and steps described above indicate certain events occurring in certain order, those of ordinary skill in the art having the benefit of this disclosure would recognize that the ordering of certain steps may be modified and that such modifications are in accordance with the 30□ variations of this invention. Additionally, certain of the steps may be performed concurrently in a parallel process when possible, as well as performed sequentially. Accordingly, embodiments are intended to exemplify alternatives, modifications, and equivalents that may fall within the scope of the claims.

1. A method for detecting a first cardiac condition, comprising:
a. providing a first quantity of 12-lead ECG data, and at least a second quantity of 12-lead ECG data from a patient;
b. constructing a 3-D representation of cardiac activity from the first and second quantities of ECG data;
c. computing values for one or more parameters based upon at least one of said 3-D representation of cardiac activity from the first quantity of ECG data;
c. computing values for said one or more parameters based upon at least one of said 3-D representation of cardiac activity from the second quantity of ECG data;
d. calculating a difference in said one or more parameters between the first quantity of ECG data and the second quantity of ECG data;
e. conducting an analysis of said calculated differences in said one or more parameters;
f. forming one or more conclusions regarding the cardiac condition of a patient based upon the analysis of said calculated differences in one or more parameters;
g. providing feedback to a healthcare provider regarding said conclusions

2. The method of claim 1, wherein said differences in one or more parameters are calculated as absolute values of said differences.

3. The method of claim 1, further comprising applying a multifactorial analysis protocol utilizing at least one of the values of the one or more parameters; and automatically drawing one or more conclusions regarding a first cardiac condition of the patient based at least in part upon the output of the multifactorial analysis protocol.

4. The method of claim 3, further comprising evaluating said calculated differences using a support vector machine.

5. The method of claim 4, further comprising using the support vector machine for multilayer analysis.

6. A system for detecting a first cardiac condition, comprising:
a. means for providing a first quantity of 12-lead EKG data, and at least a second quantity of 12-lead ECG data from a patient;
b. means for constructing a 3-D representation of cardiac activity from the first and second quantities of ECG data;
c. means for computing values for one or more parameters based upon at least one of said 3-D representation of cardiac activity from the first quantity of ECG data;
c. means for computing values for said one or more parameters based upon at least one of said 3-D representation of cardiac activity from the second quantity of ECG data;
d. means for calculating a difference in said one or more parameters between the first quantity of ECG data and the second quantity of ECG data;
e. means for conducting an analysis of said calculated differences in said one or more parameters;
f. means for forming one or more conclusions regarding the cardiac condition of a patient based upon the analysis of said calculated differences in one or more parameters;
g. means for providing feedback to a healthcare provider regarding said conclusions
